(19) United States
(12) Patent Application Publication
(10) Pub. No.: US 2015/0159126 A1
(43) Pub. Date: Jun. 11, 2015

(54) SYSTEM FOR HYDROGEN PRODUCTION AND CARBON SEQUESTRATION

(71) Applicant: Western Hydrogen Limited, Calgary (CA)

(72) Inventors: Mykola Makowsky, Calgary (CA); Guy J. Turcotte, Calgary (CA); Neil Camarta, Calgary (CA)

(21) Appl. No.: 14/405,087

(22) PCT Filed: Jun. 4, 2013
(86) PCT No.: PCT/IB2013/001135

§ 371 Date: Dec. 2, 2014

Related U.S. Application Data
(60) Provisional application No. 61/655,379, filed on Jun. 4, 2012.

(51) Int. Cl. C12M 1/107; B01D 53/84; C12M 1/00; C01B 3/10

(52) U.S. Cl. CPC C12M 21/04; C01B 3/105; B01D 53/84; C12M 47/18; C12M 29/24; C12M 43/08

(57) ABSTRACT

A system for hydrogen production and carbon sequestration includes a carbon-containing material source; a water source; a molten salt gasification reactor configured to receive a carbon-containing material, water, and a mixture of molten salts, and where in the molten salt gasifier reactor is configured to produce a gaseous stream comprising hydrogen and carbon dioxide; and an algae growth unit configured to receive the carbon dioxide.

### CROSS-REFERENCE TO RELATED APPLICATIONS

This application claims the benefit of U.S. Provisional Patent Application No. 61/655,379, filed on Jun. 4, 2012, the entire disclosure of which is hereby incorporated by reference for all purposes in its entirety as if fully set forth herein.

### FIELD

The present technology is generally related to a process for producing hydrogen and capturing carbon dioxide by algae growth.

### BACKGROUND

Steam methane reforming (SMR) is one process of hydrogen production currently in practice. It includes the conversion of natural gas (i.e. methane) to hydrogen, and it accounts for over 90% of the world's hydrogen production. The process is based upon capturing the approximately 25 wt % of hydrogen in the methane and the hydrogen in water, by converting the methane and water to synthesis gas (i.e. a mixture of H₂ and CO) over a catalyst. The process also includes the conversion of the CO to CO₂, which is then captured or released. The overall conversion efficiency of the SMR process is about 65%, as 25-30% of the methane is required for maintaining the catalysts at efficient operating temperatures. SMR systems typically also include steam generators to provide the correct steam-to-carbon ratio for generation of the synthesis gas. Thus, the SMR systems are very energy intensive to run and are not efficient systems.

Other processes for producing hydrogen include those such as the gasification of coal and petroleum coke, and electrolysis. Petroleum coke is an almost pure carbon by-product of the thermal coking process used to upgrade heavy oils. Coal and petroleum coke typically also contain one or more of sulfur, silica, and trace amounts of metals. The conventional method of producing hydrogen from coal or petroleum coke is to construct a gasification plant that produces synthesis gas. Gasification plants use air, oxygen, or steam to oxidize the coal or petroleum coke. The cost of a commercial-sized gasification plant is generally quite high. The trade-off between using oxygen and air is the cost of a cryogenic oxygen plant versus the large size of all the piping and vessels required by the air blown systems. The air blown system also produces less hydrogen per cubic foot of synthesis gas because of nitrogen dilution. With regard to electrolysis operations, they are very energy intensive are poorly suited economically for commercial scale hydrogen production.

The two primary uses of hydrogen today are for upgrading of heavy oils to commercial products and for production of fertilizer (ammonia, NH₃). While many potential non-traditional petroleum resources are known, such as bitumen, they tend to be extremely heavy and are not readily processable as fuels. Accordingly, it is necessary to add significant amounts of hydrogen to bitumen to upgrade it to the point where it can be shipped by pipeline and used as refinery feedstock. In the upgrading process and attendant hydrogen production, a number of low value products and significant emissions of green house gases (GHGs) are generated and lost to the atmosphere. With regard to the ammonia fertilizer, the industry is centered upon hydrogen production from natural gas by the SMR process described above. Therefore, the fertilizer industry tends to be located in geographic areas with high natural gas production to the exclusion of other geographic areas. Accordingly, new and greener methods of hydrogen production from renewable sources have the potential to greatly impact the production of petroleum resources and also allow for the production of fertilizer in overlooked geographical areas.

### SUMMARY

In one aspect, a system is provided for producing a gaseous stream composed primarily of hydrogen and carbon dioxide from a wide variety of materials including both renewable and non-renewable resources. The hydrogen produced may be used in a wide variety of applications, such as, but not limited to fuel cell applications and upgrading of oils. The produced carbon dioxide may be used for algae growth, which in turn may be used in further hydrogen production. In addition to the hydrogen and carbon dioxide, the gaseous stream may also contain other gases such as, but not limited to, methane, carbon monoxide, hydrogen sulfide, and water vapor.

In one aspect, a system includes an algae growth unit configured to produce algae; a molten salt gasifier (MSG) reactor including a molten salt, and configured to receive a mixture of water and at least a portion of the algae, and wherein the molten salt gasifier reactor is configured to produce a gaseous stream from the algae and water, the gaseous stream including primarily hydrogen and carbon dioxide. For example, the gaseous stream may include from about 20 mol % to about 80 mol % hydrogen or from about 80 mol % to about 20 mol % carbon dioxide. This includes where the gaseous stream includes from about 55 mol % to about 75 mol % hydrogen and from about 45 mol % to about 25 mol % carbon dioxide. In one embodiment, the gaseous stream contains from about 67 mol % to about 71 mol % hydrogen and from about 33 mol % to about 29 mol % carbon dioxide. In some embodiments, the molten salt includes sodium salts.

The molten salt reactor and the algae growth unit may be in fluid communication with one another, such that the algae growth unit is a source of at least a portion of the algae and at least a portion of the water, and the carbon dioxide that is produced in the MSG is in communication with the algae growth unit. In any of the above embodiments, the system further includes a gas/water separation subsystem configured to receive the gaseous stream from the molten salt reactor and separate the hydrogen and carbon dioxide. In any of the above embodiments, the system further includes a heat exchange system configured to convey heat generated in the molten salt reactor to the algae growth unit to maintain the temperature of the algae growth unit at a temperature favorable for algae growth. The heat exchange system may also be configured to convey heat generated from cooling at least a portion the product gas, combusting at least a portion of the product gas or purge gas, or the reaction of the product gas in a fuel cell or other electrical or heat generating unit. In any of the above embodiments, the heat exchange system is configured to convey heat generated in the molten salt reactor to pre-heat the water and algae to be received by the molten salt reactor.

In any of the above embodiments, the algae growth unit includes algae growth tanks, tubes, vats, or ponds. In any of the above embodiments, the algae growth unit may be exposed to sunlight, artificial light, or a combination of sunlight and artificial light. In any of the above embodiments, the algae growth unit includes algae growth tanks, tubes, or vats which are at least partially constructed of a transparent material that allows for passage of sunlight and/or artificial to an algae growth area.

In any of the above embodiments, the system may further include a hydrogen fuel cell configured to receive at least a portion of the hydrogen and which is configured to provide electricity.

In another aspect, a method of hydrogen production including contacting a carbon-containing material with water and a molten salt in a molten salt reactor to produce a gaseous mixture that primarily includes hydrogen and carbon dioxide; separating the gaseous mixture into purified hydrogen and purified carbon dioxide; and conveying the purified carbon dioxide to an algae growth unit for consumption by algae; wherein the molten salt includes sodium hydroxide and sodium carbonate and the carbon-containing material includes algae. In one embodiment, the method also includes conveying a portion of the algae in the algae growth unit to the molten salt reactor as at least a portion of the carbon-containing material.

In another aspect, a system includes a carbon-containing material source; a water source; a molten salt gasifier reactor configured to receive a carbon-containing material, water, and a mixture of molten salts, and where in the molten salt gasifier reactor is configured to produce a gaseous stream including primarily hydrogen and carbon dioxide; and an algae growth unit configured to receive the carbon dioxide. The molten salts may include sodium salts.

In any of the above embodiments, the algae growth unit includes algae growth tanks, tubes, or vats. In any of the above embodiments, the algae growth unit is exposed to sunlight. In any of the above embodiments, the algae growth unit includes algae growth tanks, tubes, or vats which are at least partially constructed of a transparent material that allows for passage of sunlight to an algae growth area.

In any of the above embodiments, the system may further include a hydrogen fuel cell configured to receive at least a portion of the hydrogen and configured to provide electricity.

In any of the above embodiments, the molten salt reactor and the algae growth unit may be in closed loop communication, such that the algae growth unit provides algae as the carbon-containing material and the molten salt reactor provides carbon dioxide to the algae growth unit for algae consumption and growth. Alternatively, the molten salt reactor and the algae growth unit may be in partial closed loop communication, the molten salt reactor provides carbon dioxide to the algae growth unit for algae consumption and growth.

### BRIEF DESCRIPTION OF THE DRAWINGS

FIG. 1 is a schematic representation of a basic molten salt gasifier system, according to one embodiment.

FIG. 2 is a schematic representation of a molten salt gasifier system with a number of optional embodiments, according to one or more embodiments.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

A system and method for producing a high pressure gas that includes hydrogen and carbon is described. The system and method are based upon the use of a molten salt gasifier (MSG) reactor that converts a carbon-containing material and water primarily to carbon dioxide and hydrogen. Where hydrogen is produced, it may be captured for a variety of applications, while produced carbon dioxide may be diverted to an algae growth unit. The hydrogen may be used, but not limited to, as a fuel for transportation, heating or power generation; as a feedstock in the production of chemicals or fertilizers; as a hydrogenating agent for foods, fats and oils; or for upgrading, refining and hydroprocessing of lower quality oils and heavy oils. The algae growth unit provides for fixation of the carbon through photosynthesis of the algae, and the unit provides algae which may then be used as the carbon-containing material to fuel the system.

Overall, the process is environmentally friendly, capturing the produced carbon dioxide as opposed to venting it to the atmosphere, and by using the algae that is generated, the system has the ability to become near carbon-neutral in its environmental impact, thereby potentially reducing, or eliminating, the need for extraneous hydrocarbon materials. The MSG system requires only a fuel (i.e. carbon-containing material) and water as feedstocks to produce the hydrogen and carbon dioxide. The molten salt component of the system requires very little, if any, replenishment during operation. Thus, normally wet feedstocks, such as biomass, may be ideal feedstocks for use in the system and methods.

The MSG reactor utilizes molten salts to decompose carbon-containing materials into gaseous streams of primarily hydrogen and carbon dioxide. The gaseous stream may also contain other gases such as, but not limited to, water vapor, methane, hydrogen sulfide, and/or carbon monoxide. The carbon materials in the waste may be used as fuel to provide energy for regeneration of the molten salts, and thus the system does not require much, if any, external energy input, once the system is sustained. Some features of MSG reactors have been described in, for example, U.S. Pat. Nos. 7,153,489; 7,078,012; and 6,997,012; and U.S. Patent Publication Nos. 2011/0089377 and 2011/0135565.

The gaseous stream may include from about 20 mol % to about 80 mol % hydrogen or from about 80 mol % to about 20 mol % carbon dioxide. This includes where the gaseous stream includes from about 55 mol % to about 75 mol % hydrogen and from about 45 mol % to about 25 mol % carbon dioxide. In one embodiment, the gaseous stream contains from about 67 mol % to about 71 mol % hydrogen and from about 33 mol % to about 29 mol % carbon dioxide. Other gases may also be present in lesser mol % amounts.

The MSG process occurs across a wide range of pressures, from near atmospheric to high pressure in the reactor, but with three distinct steps occurring simultaneously. Multiple reactors may be used depending on the production amount requirements and feedstock availability. The pressure inside the reactor is achieved at the front-end of the process through pressurization of the input water stream and carbon-containing material feedstock. The first step includes reacting sodium carbonate with water and carbon-containing material thereby generating sodium, carbon dioxide, and hydrogen. The second step involves reacting the sodium with water, thereby generating sodium hydroxide and hydrogen. As a result of the large quantities of water that are present in the process, this second step reaction of sodium with water occurs as the sodium metal is being generated in the first step. The third step includes reacting the sodium hydroxide with carbon and water, thereby generating sodium carbonate and hydrogen. The net chemical reactions are shown below.

Na₂CO₃+C+H₂O→2Na+2CO₂+H₂ Equation 1

2Na+2H₂O→H₂+2NaOH Equation 2

2NaOH+C+H₂O→Na₂CO₃+2H₂ Equation 3

Based upon equations 1-3, the sodium salts are not consumed in the reaction(s). The only products that are consumed are the carbon-containing material and the water. It is expected that less than 1 wt % of the sodium salts are lost during the reaction cycle and accordingly, only minor amounts will be fed into the system once the process is established and running in a given MSG reactor. The presence of hydrogen on the carbon containing compounds (e.g. carbohydrates, lipids, proteins in the biomass) will increase the yield of hydrogen in the process.

The wide range of MSG operating pressures, from near atmospheric pressure up to 2500 psig is the ability to directly produce a stream of pressurized hydrogen at the required operating pressure, reducing or eliminating the need for pressurization of the hydrogen after production.

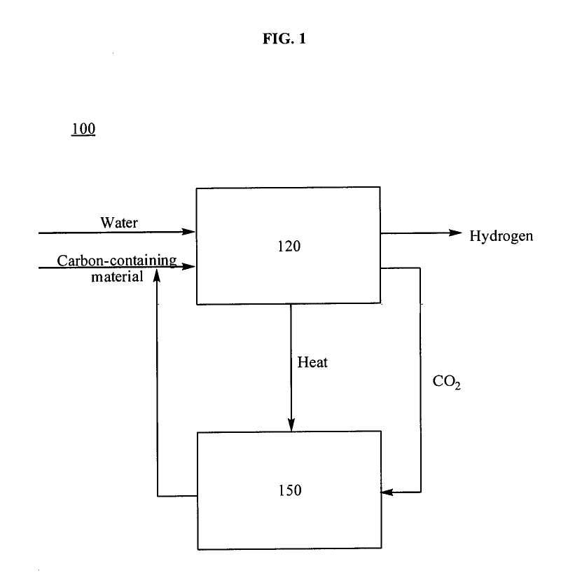

Referring now to the figures which should be read in conjunction with one another and not in isolation of each other, a number of the components of the MSG-algae growth system are described. Referring now to FIG. 1, an MSG system 100 is described schematically. The system contains two primary components, a MSG reactor 120 for conversion of a carbon-containing material and water to carbon dioxide and hydrogen, and an algae growth unit 150 for fixation of the carbon from the carbon dioxide. The MSG reactor 120 is fluidly connected to a carbon-containing material source that provides carbon-containing material (CCM), and a water source that provides water for the reaction to produce hydrogen and carbon dioxide. Within the MSG reactor is a molten salt mixture of NaOH and Na₂CO₃ which converts the carbon-containing material and water to the hydrogen and carbon dioxide. The water and carbon-containing material are supplied to the MSG reactor 120 at a near atmospheric to high pressure. The produced hydrogen and carbon dioxide can be produced at an elevated pressure so post-pressurization of the products is not required. The hydrogen may be diverted from the system for a wide variety of uses, while the carbon dioxide is diverted to the algae growth unit 150. Within the algae growth unit 150, algae consume the carbon dioxide in the presence of sunlight through photosynthesis, thereby fixing the carbon and growing new carbon-containing material (i.e. algae) for use in feeding to the MSG reactor 120. The produced gases from the reactor, or cooling water that is used to cool the reactor may all be sources of heat that may be utilized by the algae growth unit to maintain the algae at favorable algae growth temperatures.

The MSG reactor 120 includes an internal crucible that achieves temperatures from about 800° C. to about 1200° C. where the reaction between the carbon-containing material and water takes place. The internal crucible is surrounded by a cooled pressure boundary that provides temperature control of the MSG reactor 120. The crucible and cooled boundary are contained within an enclosure that is configured to contain high pressures in excess of 100 bar at the reaction temperatures. The MSG reactor 120 may also contain an inert gas source. The inert gas may be used as a control of the pressure within the crucible by providing a pressurized gas source that will not react with the carbon-containing material, the water, or the molten salt. Suitable inert gases include, but are not limited to, helium, nitrogen, or argon.

Temperatures within the MSG reactor 120 are from about 800° C. to about 1200° C. In some embodiments, the temperature is from about 850° C. to about 1000° C. In other embodiments, the temperature is about 930° C. Pressures within the MSG reactor 120 may be greater than 100 bar. For example, the pressures within the MSG reactor 120 may be from about 100 bar to about 300 bar. In some embodiments, the pressure is from about 100 bar to about 200 bar. In other embodiments, the pressure is about 135 bar to about 145 bar.

As introduced above, a significant portion, or all, of the carbon dioxide that is generated may be depressurized through a regulator or other like device and diverted to one or more algae growth units 150. The algae growth units 150 may also be connected to exogenous sources of carbon dioxide to supplement algae growth. The algae growth units 150 are also exposed to sunlight or other growth light conditions to drive the photosynthetic process of the algae. The algae in the algae growth units may also be exposed to fertilizer or other media and/or nutrients that provide for higher rates of algae growth. The algae, through photosynthesis, grow in the algae growth units, fixing the carbon from the carbon dioxide into the growing algae. Thus, the algae growth units must be of a scale such that the algae may fixate as much carbon from the carbon dioxide as possible, while providing wet algae as a fuel source to be feed to the MSG reactor 120 as a carbon-containing source material.

The algae growth unit may include any bioreactor that may be modified to contain and grow algae. For example, the bioreactor may include, but is not limited to, tanks, tubes, vats, films, or the like, such that the atmospheric conditions in contact with the algae may be controlled and appropriate levels of carbon dioxide maintained for algae growth. The bioreactors may be contained systems such as tube or containment films, or the bioreactors may be in large rooms or defined areas which contain one or more media for growing algae. Open air ponds may also be used to algae growth and carbon sequestration.

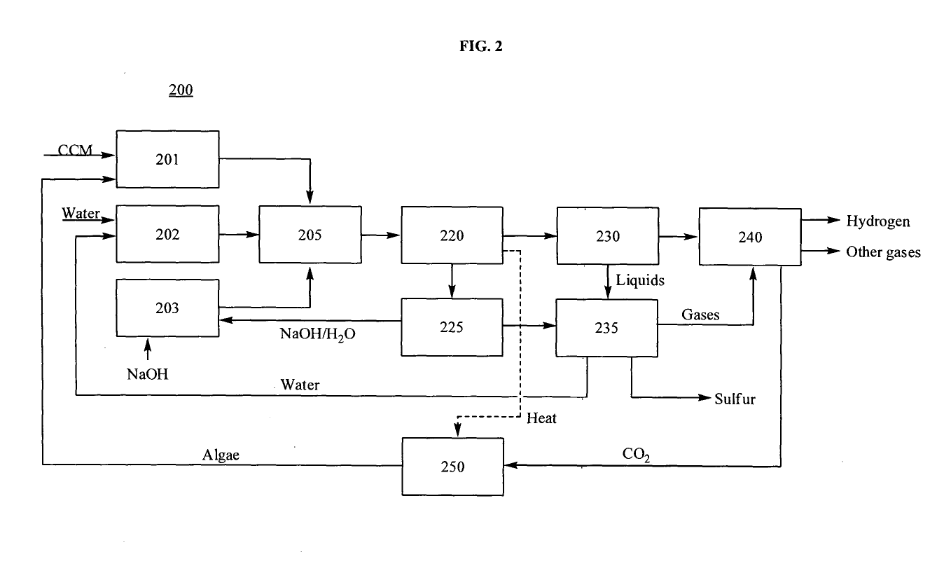

Referring now to FIG. 2, an MSG system 200 is described similar to that as described above in FIG. 1, but with several additional components present for various purposes. The system 200 includes a MSG reactor 220 for conversion of a carbon-containing material and water to carbon dioxide and hydrogen. The MSG reactor 220 is fluidly connected to a carbon-containing material (CCM) subsystem 201, a water subsystem 202, and a make-up and recycle subsystem 203. The subsystems 201, 202, 203 may optionally be connected to a feed subsystem 205 where the materials are pressurized and/or heated, and collectively supplied to the MSG reactor 220 at the proper pressure and temperature. Alternatively, the subsystems 201, 202, 203 may directly supply the reactor with the required reactants and molten salt materials. The MSG reactor 220 is fluidly connected to a carbon-containing material source that provides carbon-containing material (CCM), referred to here as the CCM subsystem 201. The water subsystem 202 provides for a water source for the reaction to produce hydrogen and carbon dioxide.

The make-up and recycle subsystem 203 may contain the molten salts from the MSG, recycled water and organic compounds, in addition to feed additives including, but not limited to glycerol for use in the MSG reactor 220. For example, the molten salts, such as, but not limited to, NaOH and Na₂CO₃ may be recovered from the MSG, and cleaned for re-introduction and recycling to the MSG.

Upon introduction of the carbon-containing material and water to the MSG 220, the reaction proceeds with the molten sodium salts to result in hydrogen and carbon dioxide production. The hydrogen and carbon dioxide may contain water vapor, the water being optionally separated in a gas/water separation subsystem 230. Any molten sodium salts saturated with impurities and other non-gaseous materials are removed from the MSG reactor 220 for recycling or disposal in an optional slag stream processor 225. Sodium salts available for recycling may be combined with the liquid removed from the gas/water separation subsystem 230 in an optional liquid cleanup subsystem 235 or recycled directly to the make-up and recycle subsystem 203. The liquid cleanup subsystem 235 separates the non-recyclable components, such as ash or sulfur, for disposal from the recyclable components, such as organic compounds, sodium salts, water, and gases. Non-recyclable components are disposed of, recyclable components are recycled to the make-up and recycle subsystem 203 and gases are introduced to the gas separation subsystem 240.

As introduced above, the MSG reactors, the produced gases from the reactors, or cooling water that is used to cool the reactor may all be sources of generated heat energy from the system, due to the high temperatures of the gasification process. Excess heat generated in the MSG reactor, or conveyed from either the produced gases or the cooling water which heated during cooling, may be captured and used in a variety of subsystems in the overall MSG-algae system. For example, the heat energy may be utilized by the algae growth unit to maintain the algae at favorable algae growth temperatures. The heat energy may also be diverted to the water subsystem or the carbon-containing material subsystem to pre-heat, or heat the respective water and carbon-containing material for introduction to the MSG reactor. Where an algae-water slurry is collected from the algae growth unit, the heat energy from the MSG reactor may be used to assist in excess water removal to achieve a desired algae:water weight ratio for introduction of the algae-water slurry to the reactor. Thus, the heat energy is not wasted, but rather it is captured to assist in achieving or maintaining a self-sustaining reaction for the generation of hydrogen. The capture may be through venting, heat exchangers, or other heat transfer mechanisms.

The carbon-containing material subsystem 201 includes a heated tank that raises the temperature of the material such that it will flow as a liquid or slurry into the MSG reactor. Such temperatures may range from about 25° C. up to temperatures without decomposing the algae material. For example, such temperatures may be from about 25° C. to 400° C. In other embodiments, the temperatures may be from about 30° C. to about 300° C. In the one embodiment, the algae slurry or liquid is be heated to a temperature just below the temperature at which thermal decomposition of the algae begins, prior to feeding the algae slurry/liquid to the MSG reactor. Alternatively, the algae can be heated from the algae growth unit temperature up to an operating temperature in the reactor. The carbon-containing material is in a closed tank at atmospheric pressure in the subsystem 201, prior to introduction to the MSG reactor 220. The subsystem 201 may contain a pressurization and pumping system for the introduction of the material to the MSG reactor 220 at a pressure from about 100 bar to about 300 bar. The subsystem 201 may also contain a feed preparation system to process the CCM into a pumpable medium that may be introduced to the MSG reactor 220 at elevated pressures. For example, the feed preparation system may include grinding, mashing, cutting, and pumping systems for preparation of the biomass prior to feeding to the reactor.

The water subsystem 202 has a water source from both exogenous sources and system-recycled sources. There are two primary uses of water uses in the pilot plant. The first is to provide steam to the MSG reactor 220, and the second is for cooling of the MSG reactor 220. Although the subsystem 202 may include a tank at atmospheric pressure for preparation, the subsystem 202 also includes a pressurization system that raises the pressure of the water to about 100 bar to about 300 bar. The subsystem may also include preheating treatment to raises the temperature of the water to close to the temperatures achieved in the MSG reactor 220. For example, the water may be pre-heated to steam and then to superheated steam. Illustrative temperatures are from about 100° C. to about 400° C. The subsystem 202 may also contain a water treatment system, such as filtration or reverse osmosis, to ensure that any fresh or recycled water meets process equipment water specifications.

The gases from the MSG reactor 220 enter the gas/water separation subsystem 230, at high temperature and pressure. In addition to the hydrogen and carbon dioxide, the gases may contain acids, such as hydrogen sulfide, or nitric acid, as well as other inorganic carbon sources such as carbon monoxide. The gases may be treated in the gas/water separation subsystem with a base spray to neutralize the acids, with an adsorbent, or with a catalytic system to remove the impurities. For example, where the gases contain hydrogen sulfide, the gases may be treated with a dilute spray of a base such as, but not limited to NaOH, KOH, NaHCO₃, KHCO₃, NH₃, K₂O, or Na₂O. Other oxidants may be added to the spray as well. For example, hydrogen peroxide may be added to the spray either alone or in combination with any of the bases. Alternatively or in addition, the gases may be exposed to a sulfur adsorbent material such as zinc oxide, or with a catalytic, sulfur oxidation material that converts hydrogen sulfide to elemental sulfur. In the gas/liquid separation subsystem, the temperature of the gas may be reduced to condense the water for removal. The gases may then be separated or transferred to an optional gas cleanup subsystem 240. The gas/liquid separation subsystem may be located prior to or after the optional gas clean up subsystem 240. The gas cleanup subsystem 240 may provide for separation of gasses such as hydrogen, carbon dioxide, carbon monoxide, hydrogen sulfide, and methane produced by the MSG reactor 220 into purified components.

In the gas cleanup subsystem, the gases may be extracted with water under high pressure. Both hydrogen sulfide and carbon dioxide have high solubility in water at high temperature. Accordingly, by extraction with water, purified hydrogen may be obtained. The purified hydrogen may be greater than 80% pure. In some embodiments, the hydrogen is greater than 90%. In other embodiments, the hydrogen is greater than 95% pure. In yet other embodiments, the hydrogen is greater than 99% pure. The purified hydrogen may be from 70% to 100% pure. The pressure on the water extracts is then reduced and the carbon dioxide is released from the water for capture and venting to the algae growth unit 250. Other gas cleanup process in the subsystem 240 may include amine scrubbing or methanation reactions of the hydrogen to remove other trace materials and produce a high purity hydrogen stream. The gas clean up subsystem may also include cryogenic purification of the gases, with cryogenic cooling of the gas mixture and controlled heating to distill and collect each gas as it evolves from the cooled mass. The gas clean up subsystem may also include purification using a hydrogen separation membrane, solvent or amine based absorption or other conventional gas separation technologies.

The reactor feed may contain a certain amount of inorganic materials. Over time these will accumulate in the molten salt bed and would need removal. Thus, the slag stream processor 225 is connected to the MSG reactor 220 to receive spent molten salt for regeneration. The molten salt that enters the slag stream processor 225 may contain water, sulfur, metals, and the like. If the amount of inorganic material requires frequent removal, a rotating slag tap may be used to remove a portion of the bed. After being dissolved in water, the solids will be removed and the dissolved sodium salts recycled to the make-up and recycle subsystem 203 and re-introduced to the reactor with the water.

The liquid cleanup subsystem 235, is an optional subsystem, which may receive the materials from the slag stream processor 225, and the liquid materials from the gas/water separation subsystem 230. In the liquid cleanup subsystem 235, dissolved hydrogen and carbon dioxide in the liquid may be liberated and then fed back to the gas cleanup subsystem 240 (if present), with water being recycled back to the water subsystem 202 or the make-up and recycle subsystem 203, and any sulfur that has carried through from the gas water separation subsystem may also be separated. The system 235 may also include filters for purification of the water before recycling to the water preparation subsystem 202 or the make-up and recycle subsystem 203.

The MSG reactor 220 includes an internal crucible that achieves temperatures from about 800° C. to about 1200° C. where the reaction between the carbon-containing material and water takes place. The internal crucible is surrounded by a cooled pressure boundary that provides temperature control of the MSG reactor 220. The crucible and cooled boundary are contained within an enclosure that is configured to contain high pressures in excess of 100 bar at the reaction temperatures. The MSG reactor 220 may also contain an inert gas source. The inert gas may be used as a control of the pressure within the crucible by providing a pressurized gas source that will not react with the carbon-containing material, the water, or the molten salt. Suitable inert gases include, but are not limited to, helium, nitrogen, and/or argon.

Recovery of the hydrogen and carbon dioxide from the gas/water separation subsystem 230 may proceed via the optional gas cleanup subsystem 240. In the gas cleanup subsystem 240, the hydrogen, carbon dioxide, and other minor constituent gases (e.g. carbon monoxide) are separated into their purified constituents. Technologies for gas separation include, but are not limited to, pressure swing adsorption (PSA), cryogenic recovery, zinc oxide sulfur adsorption, flashing and hydrogen separation membranes. Suitable hydrogen separation membranes include, but are not limited to, ceramic, ‘cermat’ or metallic hydrogen ion transport membranes or ceramic, ‘cermat’ or metallic hydrogen permeable membranes. In the 230 and 240 subsystems, the pressure from the reactor is maintained, such that when the purified hydrogen and carbon dioxide gases emerge from the gas cleanup subsystem 240, the gases remain at the MSG reactor 220 operating pressures at which they may be contained or otherwise diverted to an end use. For example, the hydrogen and carbon dioxides may be contained in pressurized canisters or tankers for a wide variety of uses. Additionally, contaminant materials, such as sulfur, may be removed from the system via the liquid cleanup subsystem where the sulfur is separated and diverted for recovery.

A significant portion, or all, of the carbon dioxide that is generated may be depressurized through a regulator or other like device and diverted to one or more algae growth units 250. Alternatively, the carbon dioxide can remain in solution instead of being separated and be diverted to a water storage tank. The CO2 infused water can then be fed to the algae growth unit. Algae growth units 250 are in fluid communication with a carbon dioxide outlet from either the gas cleanup subsystem 240, or the gas/water separation subsystem 230. Alternatively, the algae growth units 250 may be fed with exogenous sourced carbon dioxide to support or replace the carbon dioxide feed from the subsystems. The algae growth units 250 are also exposed to sunlight or artificial light to drive the photosynthetic process of the algae. The algae, through photosynthesis, grows in the algae growth units, fixing the carbon from the carbon dioxide into the growing algae. Thus, the algae growth units must be of a scale such that the algae may fixate as much carbon from the carbon dioxide as possible, while providing wet algae as a fuel source to be feed to the MSG reactor 210 as a carbon-containing source material.

The algae growth unit may include any bioreactor that may be modified to contain and grow algae. For example, the bioreactor may include, but is not limited to, tanks, tubes, vats, films, or the like, such that the atmospheric conditions in contact with the algae may be controlled and appropriate levels of carbon dioxide maintained for algae growth. The bioreactors may be contained systems such as tube or containment films, or the bioreactors may be in large rooms or defined areas which contain one or more media for growing algae. Open air ponds may also be used to algae growth and carbon sequestration.

The MSG reactor 220 may also be used as a source of heat for the algae growth unit 250. Heat exchangers on the MSG reactor 220 may capture heat and transfer it to the algae growth unit 250, thereby maintaining the algae at temperature conditions favorable for algae growth. For example, temperatures from about 25° C. to about 40° C. are good temperature growing conditions for algae, although these temperatures may vary depending on the particular algae being grown.

The MSG reactor is configured to produce high pressure hydrogen without the need for an oxygen plant or hydrogen compression. This increases the efficiency of the process as the post-production compression of hydrogen in other hydrogen generation system can require as much as 20% of the total input energy to the process. The high pressure hydrogen may be produced at pressures of 100 bar, or higher. For example, the high pressure hydrogen may be produced at pressures from 100 bar to about 200 bar. In some embodiments, the high pressure hydrogen is produced at a pressure from about 120 bar to about 150 bar.

The MSG reactor may be configured to convert a wide variety of carbon-containing materials and water to hydrogen and carbon dioxide. For example, carbon-containing materials may include, but is not limited to, natural gases, coal, oil, and the like. However, such sources are typically high cost and while they do work, conversion of traditionally low value materials, or renewable materials, is desired. Low value carbon-containing materials may include, but is not limited to, glycerol from biodiesel production, pitch, coke, asphaltene, biowaste, and biomass. For example, biomass may include, but is not limited to, sawgrass, straw, silage, wood chips, algae, water sludge, sewage treatment solids, food wastes, and the like. In one embodiment, the fuel source is algae. As indicated above, the present system incorporates an algae growth unit to take advantage of this material as a potential carbon-containing material for maintaining the reaction to produce hydrogen and carbon dioxide.

The MSG reactor may be configured to accommodate large amounts of water from the water source. For example, the MSG reactor may be configured to accommodate up to forty times the stoichiometric ratio of water to carbon-containing material. Accordingly, the MSG-algae system, using only a carbon-containing material and water, is an ideal system for the use of wet algae, or any wet biomass, as a feedstock to the system. Thus use of the wet fuel sources eliminates the need for drying of the algae, or any other type of biomass, prior to introduction to the MSG reactor, thereby significantly avoiding energy costs that trouble other hydrogen production systems. Ratios of water to algae may be adjusted through process such as filtering or centrifuging, which are less energy intensive than drying of the algae. For example, a 90:10 mass ratio of water:algae is approximately a ten-fold stoichiometric excess of water. While additional energy is needed in higher water loadings to maintain the MSG at operating temperatures, balancing of the feed ratios and rates can enable establishment of a self-sustaining system of hydrogen production. Accordingly, with an algae feed from the algae growth unit to the MSG reactor, a self-sustaining system, with additional water influx as needed, may be attained.

The systems may be run as a closed loop system. In other words, once the system is running, the hydrogen production is maintained by feeding algae to the MSG reactor at pressure and recovering the carbon dioxide for use in growing the algae. The carbon cycle is maintained within the closed loop of the system, while the hydrogen that is generated is removed and water is added.

The system may alternatively be operated as a partial closed loop system, where other biomass or materials may be added to supplement the algae from the algae growth unit during times of lower algae production or high demand for hydrogen. Such a partial closed loop system operates as with the fully closed loop system, however, the loop may be broken to allow for other carbon-containing materials to be added. For example, biomass waste such as straw, silage, hay, wood chips, and exogenous algae (such as from vacuuming from the ocean or other lake source) may be added to the system. Alternatively, where there is a need for oil from algae, or the commodity price of algae oil is such that it makes economic sense to recover it instead of using it as a carbon-containing material, the algae may be removed from the algae growth units and used for oil production, while the MSG is fed with other carbon-containing source materials such as oil, natural gas, biomass, and the like.

The heat from the reactor may also be diverted through heat exchange to maintain the temperature of the algae growth unit at algae growing temperatures, or from the reactor to the water processing subsystem to pre-heat the water, or from the reactor to the carbon-containing material subsystem to pre-heat the carbon-containing material, or any two or more of these heating processes. Depending on the type of algae being grown, the optimal temperature for growth may fluctuate and more or less heat may be required.

Any of the above systems may further include one or more hydrogen fuel cells. The hydrogen fuel cells are configured to receive a portion of the product gases, which include hydrogen, that are produced and convert the hydrogen to electrical power. The power thus generated could be used to power any one or more of the systems and subsystems described above including pumps, lights, heating units, and the like, or used for exogenous electrical consumption outside the system. The systems may be scaled for hydrogen production to account for both intended end uses of the hydrogen as well as use in the fuel cell(s). Alternatively, or in addition, to the fuel cell, the system may include a fired burner to capture the energy from the hydrogen for use in the system, or to combust other gases generated in the system. The flue gasses, including carbon dioxide, from such combustion may be re-captured and directed to the algae growth systems.

In another aspect, a method of hydrogen production is provided. Much of the method has been described above in terms of how the system operates, however, other aspects of operation are described below. The method includes contacting a carbon-containing material with water and a molten salt in a molten salt reactor to produce a gaseous mixture of hydrogen and carbon dioxide. This may be effected in either the simplified system described in FIG. 1, or the more complex system and subsystems described by FIG. 2. The gaseous mixture that is then prepared in the reactor is separated into purified hydrogen and purified carbon dioxide. It should be noted that while high purity hydrogen and carbon dioxide may be prepared with the systems and methods, the term “purified” simply refers to a separation of the materials, where a majority of the “purified” gas is the one designated. Thus, purified hydrogen refers to a hydrogen product that is higher in hydrogen content than the hydrogen as produced in the reactor. Or, the term purified carbon dioxide refers to a carbon dioxide product that is higher in carbon dioxide content than the carbon dioxide as produced in the reactor. The method also includes conveying the carbon dioxide to an algae growth unit for consumption by algae. In such methods, the molten salt includes sodium hydroxide and sodium carbonate.

In one embodiment of the method, a portion of the algae that is produced in the algae growth unit is conveyed to the molten salt reactor. Thus, the algae forms at least a portion of the carbon-containing material that is fed to the molten gas reactor. Upon start up of such a system, the algae will not have had ample opportunity to become established, and growth rates may not have reached acceptable levels in the algae growth unit. Accordingly, at the beginning of the method, other carbon-containing material sources may be used such as natural gas, coal, coke, and other biomass to be converted to hydrogen and carbon dioxide in the reactor. As the algae growth increases, the system may be converted to run at least partial on the algae that is collected for conveyance to the reactor. The algae amounts fed to the reactor may be supplemented at any time with additional biomass, natural gas, coal, coke, and the like to meet hydrogen demand or account for slow downs in algae growth. However, in one embodiment, the amount of algae produced in the algae growth unit is sufficient to meet the needs of the hydrogen production and the system will approach a self-maintaining level with regard to the amount of carbon-containing material needed for the process.

The present invention, thus generally described, will be understood more readily by reference to the following examples, which are provided by way of illustration and are not intended to be limiting of the present invention.

### EXAMPLES

### Example 1

Example 1 is a general description of operation of the system. A carbon-containing material reservoir is charged, at ambient temperature, with carbon-containing material, at atmospheric pressure, to be fed to the MSG reactor. The water preparation subsystem is also then charged with a water source. Upon initiation of the reactor, the water and carbon-containing material are pressurized to about 100 bar. The water is heated in a steam generator and superheater, and then charged as a heated steam charge to the MSG reactor. Water is also used for cooling of the MSG reactor to maintain the wall temperatures below material temperature limits. The sodium makeup subsystem is charged with a dilute solution of sodium hydroxide which is used to replenish minor losses of sodium during hydrogen production. The reactor pressure may be supplemented by pumping in pressurized inert gases to maintain the pressure at operating conditions. The crucible of the MSG reactor is maintained at a temperature of approximately 930° C. where the conversion of the carbon-containing material and water is converted to hydrogen and carbon dioxide.

After generation of the hydrogen and carbon dioxide, the crude gases are transferred to the gas-water separation subsystem. In this subsystem, the crude gases may be contacted with a spray of dilute sodium hydroxide and hydrogen peroxide to neutralize any acid gases that are produced in the MSG as a result of non-carbon or hydrogen material content (e.g. sulfur-containing materials). Alternatively, adsorbent materials such as zinc oxide may be used to sequester acid components such as hydrogen sulfide, or the hydrogen sulfide may be catalytically converted to elemental sulfur. Through temperature reduction in the gas/water separation subsystem, the water is then condensed from the gases to provide clean separation of water vapor that may be carried through the MSG reactor. The water that is recovered is then recycled back to the water preparation subsystem or the make-up and recycle subsystem.

The solids and molten materials from the MSG reactor are then sent to the slag stream processor to remove inorganic materials that will build up in the molten salt material. The molten salt is dissolved in water, the solids removed by filtration and the dissolved molten salts are re-introduced to the MSG reactor.

The hydrogen and carbon dioxide gas mixture is then transferred to a gas cleanup system where the gases are separated by cryogenic separation, the carbon dioxide being cooled to either a liquid or solid while the hydrogen remains as a gas that is then collected. Alternatively, the gases may be separated by pressure swing adsorption, flashing or using a hydrogen separation membrane. The carbon dioxide is then fed to an algae growth unit for sequestration.

After the algae growth has produced enough algae that it may be harvested from the growth unit, the algae is removed as a wet solid that is then introduced to the MSG reactor as a carbon-containing material to sustain the process. Additional water is added, as is a dilute solution of sodium hydroxide to maintain the molten salt concentration in the reactor.

### Example 2

Example 2 is based upon calculations for a pilot plant scale operation of the system. At startup of the system, natural gas (approximately 1,500 kg/day) and water (6.2 m³/day) are introduced to an MSG reactor at 930° C. having a molten salt mixture of NaOH and Na₂CO₃. The MSG reactor will produce about 480 kg/day of hydrogen and about 3990 kg/day of carbon dioxide. The hydrogen is obtained at a pressure of about 137 to about 140 bar. The carbon dioxide at a similar pressure is then down regulated in pressure and is then sent to the algae growth unit, which includes a series of tubes of water with algae and nutrients, where the algae will consume the carbon dioxide at a rate of about 1 g to about 4 g of carbon per liter per day, producing about 2 g to about 9 g of algae per liter per day. After algae growth is sustainable, algae is then collected, and sent to the MSG reactor wet (because the MSG conversion uses carbon-containing material and water as feedstocks, wet algae is well-suited as a carbon-containing material and water supply). Eventually, with sufficient algae production, the system may approach or attain a self-sustaining status.

### Example 3

Example 3 is based upon calculations for a pilot plant scale operation of the system. At startup of the system, biomass of sawgrass, algae, or wood (approximately 4480 kg/day) and water (6.7 m³/day) are introduced to an MSG reactor at 930° C. having a molten salt mixture of NaOH and Na₂CO₃. The MSG reactor will produce about 480 kg/day of hydrogen and about 8950 kg/day of carbon dioxide. The hydrogen is obtained at a pressure of about 100 bar. The carbon dioxide at a similar pressure is then down regulated in pressure and is then sent to the algae growth unit, which includes a series of tubes of water with algae and nutrients, where the algae will consume the carbon dioxide at a rate of about 1-4 g of carbon per liter per day, producing about 2-9 g of algae per liter per day. The algae that is produced may then be used either for exogenous purposes, i.e. to produce plant-based fertilizers, algae oil, or the like, with other biomass then be used to sustain the hydrogen and carbon dioxide production in the MSG reactor, or after algae growth is sustainable, the algae is collected and sent to the MSG reactor wet, as above.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A system comprising:
an algae growth unit configured to produce algae;
a molten salt gasifier reactor comprising a molten salt, the molten salt gasifier reactor configured to receive a mixture of water and at least a portion of the algae; and
wherein:
the molten salt gasifier reactor is configured to produce a gaseous stream from the algae and water, the gaseous stream comprising hydrogen and carbon dioxide.

2. The system of claim 1, wherein the molten salt reactor and the algae growth unit are in communication, such that the algae growth unit is a source of at least a portion of the algae and at least a portion of the water, and the carbon dioxide that is produced is in communication with the algae growth unit.

3. The system of claim 1 further comprising a gas/water separation subsystem configured to receive the gaseous stream from the molten salt reactor and separate the hydrogen and carbon dioxide.

4. The system of claim 1 further comprising a first heat exchange system configured to convey heat generated in the molten salt reactor to the algae growth unit to maintain the temperature of the algae growth unit at a temperature favorable for algae growth.

5. The system of claim 4 further comprising a second heat exchange system configured to convey heat generated in the molten salt reactor to pre-heat the water and algae to be received by the molten salt reactor.

6. The system of claim 1, wherein the algae growth unit comprises algae growth tanks, tubes, vats or ponds.

7-9. (canceled)

10. The system of claim 1, wherein the gaseous stream consists essentially of hydrogen and carbon dioxide.

11. The system of claim 1 further comprising a hydrogen fuel cell configured to receive at least a portion of the hydrogen and configured to provide electricity and heat.

12. A method of hydrogen production comprising:
contacting a carbon-containing material with water and a molten salt in a molten salt reactor to produce a gaseous mixture of hydrogen and carbon dioxide;
separating the gaseous mixture into purified hydrogen and purified carbon dioxide; and
conveying the purified carbon dioxide to an algae growth unit for consumption by algae;
wherein: the molten salt comprises sodium hydroxide and sodium carbonate and the carbon-containing material comprises algae.

13. The method of claim 12 further comprising conveying a portion of the algae in the algae growth unit to the molten salt reactor as at least a portion of the carbon-containing material.

14. A system comprising:
a carbon-containing material source;
a water source;
a molten salt gasifier reactor configured to receive a carbon-containing material, water, and a mixture of molten salts, and where in the molten salt gasifier reactor is configured to produce a gaseous stream comprising hydrogen and carbon dioxide; and
an algae growth unit configured to receive the carbon dioxide.

15. The system of claim 14, wherein the carbon-containing material comprises coke, coal, natural gas, or biomass.

16. The system of claim 15, wherein the carbon-containing material comprises methane, ethane, propane, or butane.

17. The system of claim 15, wherein the carbon-containing material comprises biomass.

18. (canceled)

19. The system of claim 14, wherein the carbon-containing material comprises algae.

20-24. (canceled)

25. The system of claim 14 further comprising a gas/water separation subsystem configured to receive the gaseous stream from the molten salt reactor and separate hydrogen and carbon dioxide from the gaseous stream.

26-27. (canceled)

28. The system of claim 14, wherein the molten salt reactor and the algae growth unit are in closed loop communication, such that the algae growth unit provides algae as the carbon-containing material and the molten salt reactor provides carbon dioxide to the algae growth unit for algae consumption and growth.

29. The system of claim 14, wherein the molten salt reactor and the algae growth unit are in partial closed loop communication, the molten salt reactor provides carbon dioxide to the algae growth unit for algae consumption and growth.

30. (canceled)

31. The system of claim 14, wherein the gaseous stream consists essentially of hydrogen and carbon dioxide.

32. The system of claim 14 further comprising a hydrogen fuel cell configured to receive at least a portion of the hydrogen and configured to provide electricity and heat.
